(19) United States
(12) Patent Application Publication
(10) Pub. No.: US 2022/0152292 A1
(43) Pub. Date: May 19, 2022

(54) AUTOMATIC PRESSURE CONTROL DEVICE, SYSTEM, AND METHOD

(71) Applicant: Drägerwerk AG & Co. KGaA, Lubeck (DE)

(72) Inventor: Edward Holdgate, IV, Sandwon, NH (US)

(21) Appl. No.: 17/527,132

(22) Filed: Nov. 15, 2021

Related U.S. Application Data
(60) Provisional application No. 63/114,890, filed on Nov. 17, 2020.

(51) Int. Cl. A61M 5/148; A61M 5/172; A61B 5/022

(52) U.S. Cl. CPC A61M 5/1486; A61M 5/1723; A61M 2005/14513; A61M 2005/1726; A61B 5/02233

(57) ABSTRACT

A device, a system and a method for automatic pressure control are disclosed. The pressure control device includes one or more processors and a valve with a first port, a second port, and a third port, with the first port connected to an air pump, the second port connected to a non-invasive blood pressure (“NIBP”) cuff worn by a patient, and the third port connected to a pressure-providing element pressurizing an IV fluid container. The one or more processors control the valve to adjust airflow between the air pump and the NIBP cuff, and also to adjust airflow between the air pump and the pressure-providing element pressurizing the fluid container. By controlling the valve and the connected air pump, the system automates the processes of regulating fluid flow out of the container and non-invasively measuring the patient's blood pressure.

### CROSS REFERENCE TO RELATED APPLICATION

Priority is hereby claimed under 35 U.S.C. § 119(e) to U.S. Provisional Application No. 63/114,890, entitled “NIBP Monitoring and Control System on Saline/Medical Fluid Pressure Bag,” filed Nov. 17, 2020, currently pending, the entire contents of which are hereby incorporated herein by reference as if expressly set forth herein.

### TECHNICAL FIELD

The present disclosure relates generally to the field of automated pressure monitoring and control in a patient care environment. More particularly, the present disclosure relates to an automatic pressure control device, system, and method used in non-invasive blood pressure measurement process and intravenous infusion process in patient care.

### DESCRIPTION OF RELATED ART

Intravenous (“IV”) injection or infusion is a commonly used medical procedure. One example of IV infusion is to use an infusion pump that delivers IV fluid (often with nutrients or medications) into a patient's body in controlled manners. Required to be operated by a trained caregiver, the infusion pump automatically controls fluid delivery at pre-programmed rates and intervals. According to the U.S. Food and Drug Administration, safety issues related to infusion pumps (e.g., user error, deficiencies in device design and engineering) can cause significant implications for patient safety.

Another example of IV infusion is to use an IV line connected to a sealed container (e.g., an IV bag) filled with fluid and at an end of the IV line, an IV catheter that is inserted into the vein of the patient. The catheter allows the fluid to be injected into the vein of the patient. It does not require an electronic device and, therefore, is low cost and more commonly used. In clinical settings, the IV bag filled with fluid often requires an external pressure-providing element (e.g., an external pressure bag) that wraps around it to apply sufficient pressure to the IV bag. As such, the fluid will be “squeezed” out of the IV bag and delivered into the patient's body via the catheter. Commonly, the external pressure bag is further equipped with a manually operated hand pump, e.g., a rubber inflation bulb that is squeezed by a caregiver to inflate the pressure bag with air, until a proper pressure is reached.

Over time, the fluid flows out of the IV bag and the pressure provided by the external pressure bag reduces due to the volume decrease of the remaining fluid. Therefore, the flow rate of the IV fluid may change due to the insufficient pressure provided by the external pressure bag. When the pressure further drops, the delivery of the IV solution may be slowed, interrupted, or even paused. To avoid this, caregivers are burdened with periodically monitoring the pressure of the external pressure bag and manually pumping it up by squeezing the rubber inflation bulb. This cycle of manual pumping-up needs to be repeated numerous times until the IV infusion process completes. In some clinical settings, caregivers are often required to monitor and adjust the bag pressure every five minutes to an hour, which significantly burdens them and adversely affects the clinical workflow. Simultaneously, caregivers also typically need to monitor and/or measure various patient parameters such as cardiac output, blood pressure measured non-invasively as by a Non-Invasive Blood Pressure (“NIBP”) apparatus, respiration, neurological parameters, blood glucose, or body temperature, to list a few examples. While some parameters can be continuously monitored via sensors that are semi-permanently attached to the patient (e.g., cardiac output or respiration), other parameters (e.g., NIBP) may need to be measured periodically and may require the caregiver to manually obtain and record the measurements.

There exists a need to adjust pressure applied to the IV bag to provide a desired rate of fluid delivery throughout the IV infusion process, regardless of changes in the bag's contents during the course of the process. There also exists a need to have an automatic pressure control on the external pressure bag or other pressure-providing element that pressurizes the IV bag, to reduce or eliminate the requirement for repeated manual adjustment by caregivers. A further benefit could be realized if the same system that regulated pressure on the IV bag also automated measurements of the patient's blood pressure by a non-invasive cuff, since this task also requires repeated intervention by caregivers if done manually.

### SUMMARY

In one embodiment of the present disclosure, a pressure control device may include a controllable multi-port valve with a first port, a second port, and a third port, where the first port is connected to an air pump, the second port is connected to a non-invasive blood pressure (“NIBP”) cuff, and the third port is connected to a pressure-providing element pressurizing a fluid container. The pressure control device further includes one or more processors configured to control the ports of the valve to adjust a first airflow between the air pump and the NIBP cuff and a second airflow between the air pump and the pressure-providing element.

The present disclosure further provides a pressure control system including a controllable multi-port valve with a first port, a second port, and a third port. The first port is connected to an air pump, the second port is connected to an NIBP cuff worn by a patient, and the third port is connected to a pressure-providing element pressurizing a fluid container. The pressure control system further includes one or more processors configured to non-invasively measure the blood pressure of the patient by controlling the valve to adjust an airflow between the air pump and the NIBP cuff, and control the pressure provided to the fluid container by controlling the valve to adjust an airflow between the air pump and the pressure-providing element.

The present disclosure further provides a computer-implemented method for pressure control. The method may be stored as instructions on a non-transitory computer-readable medium. When executed, the instructions may cause a machine to perform the method. The method includes, but is not limited to, the following operations: controlling a first port, a second port, and a third port of a controllable multi-port valve; operating an air pump connected to the first port; adjusting a first airflow between the first port and the third port to operate a pressure-providing element pressurizing a fluid container; recognizing a triggering event for a blood pressure measurement; adjusting a second airflow between the first port and the second port to inflate, then deflate, an NIBP cuff worn by a patient; receiving signals from an NIBP sensor associated with the NIBP cuff; and calculating a blood pressure of the patient by analyzing the signals.

These and other aspects of the present disclosure are more fully described herein with reference to the accompanying figures.

### BRIEF DESCRIPTION OF THE DRAWINGS

Illustrative embodiments of the subject matter claimed below will now be disclosed. In the interest of clarity, not all features of an actual implementation are described in this specification. It will be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

FIG. 1 is a schematic diagram of an example physiological monitoring system for automatic pressure control according to one embodiment of the present disclosure.

FIG. 2 is a schematic diagram of an example automatic pressure control system according to one embodiment of the present disclosure.

FIG. 3 is a block diagram of an example automatic pressure control system according to one embodiment of the present disclosure.

FIG. 4 is a block diagram of an example automatic pressure control device in connection with a host device according to one embodiment of the present disclosure.

FIG. 5 is a schematic diagram of the example automatic pressure control device in accordance with FIG. 4 according to one embodiment of the present disclosure.

FIG. 6 is a block diagram of another example automatic pressure control device in connection with a host device according to one embodiment of the present disclosure.

FIG. 7 is a schematic diagram of the example automatic pressure control device in accordance with FIG. 6 according to one embodiment of the present disclosure.

FIG. 8 is a flowchart of a method of automatic pressure control according to one embodiment of the present disclosure.

### DETAILED DESCRIPTION

The following disclosure is presented to provide an illustration of the general principles of the present invention and is not meant to limit, in any way, the inventive concepts contained herein. Moreover, the particular features described in this section can be used in combination with the other described features in each of the multitude of possible permutations and combinations contained herein.

All terms defined herein should be afforded their broadest possible interpretation, including any implied meanings as dictated by a reading of the specification as well as any words that a person having skill in the art and/or a dictionary, treatise, or similar authority would assign particular meaning. Further, it should be noted that, as recited in the specification and in the claims appended hereto, the singular forms “a,” “an,” and “the” include the plural referents unless otherwise stated. Additionally, the terms “comprises” and “comprising” when used herein specify that certain features are present in that embodiment, but should not be interpreted to preclude the presence or addition of additional features, components, operations, and/or groups thereof.

The following disclosure is intended to be read in connection with the accompanying drawings, which are to be considered part of the entire written description of the invention. The drawing figures are not necessarily to scale, and certain features of the invention may be shown exaggerated in scale or in somewhat schematic form in the interest of clarity and conciseness. In this description, relative terms such as “horizontal,” “vertical,” “up,” “down,” “top,” “bottom,” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both moveable or rigid attachments or relationships, unless expressly described otherwise, and includes terms such as “directly” coupled, secured, etc. The term “communicatively coupled” describes a connection or coupling through which something movable, such as a substance or a signal, may be caused to flow from one location to another.

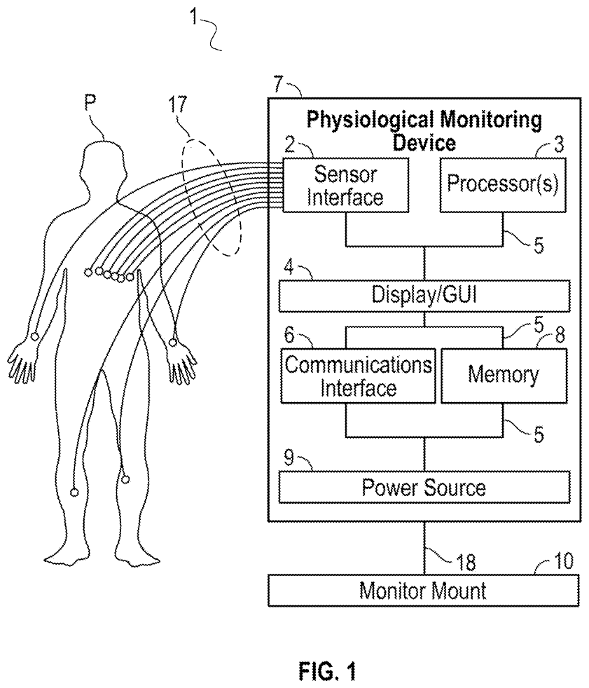

FIG. 1 is a schematic diagram of an example physiological monitoring system 1 for automatic pressure control according to one embodiment of the present disclosure. As shown in FIG. 1, the physiological monitoring system 1 includes a physiological monitoring device 7 capable of receiving physiological data from various sensors 17 connected to a patient P, and a monitor mount 10 to which the physiological monitoring device 7 is removably mounted or docked.

In general, it is contemplated by the present disclosure that the physiological monitoring device 7 and the monitor mount 10 include electronic components or electronic computing devices operable to receive, transmit, process, store, and/or manage patient data and information associated performing the functions of the system, which encompasses any suitable processing device adapted to perform computing tasks consistent with the execution of computer-readable instructions stored in a memory or a computer-readable recording medium.

Further, any, all, or some of the computing devices in the physiological monitoring device 7 and the monitor mount 10 may be adapted to execute any operating system, including Linux®, UNIX®, Windows Server®, etc., as well as virtual machines adapted to virtualize execution of a particular operating system, including customized and proprietary operating systems. The physiological monitoring device 7 and the monitor mount 10 are further equipped with components to facilitate communications with other computing devices over one or more network connections, which may include connections to local and wide area networks, wireless and wired networks, public and private networks, and any other communication network enabling communication in the system.

As shown in FIG. 1, the physiological monitoring device 7 may be, for example, a patient monitor implemented to monitor various physiological parameters of the patient P via the sensors 17. The physiological monitoring device 7 may include a sensor interface 2, one or more processors 3, a display/graphical user interface (“GUI”) 4, a communications interface 6, a memory 8, and a power source 9. The sensor interface 2 can be implemented in software or hardware and used to connect via wired and/or wireless connections to one or more physiological sensors and/or medical devices for gathering physiological data from the patient P.

The data signals from the sensors 17 may include, for example, data related to non-invasive blood pressure (“NIBP”) measurements and electrocardiogram (“ECG” or “EKG”) readings, as well as non-invasive measurements of peripheral oxygen saturation (“SpO₂”), temperature, end-tidal carbon dioxide (“ETCO₂”), apnea detection, and other similar physiological measurements. Furthermore, the patient P may also be under a medical procedure, for example, an IV injection/infusion process. Optionally, an intravenous drip rate control system or other injection/infusion control systems may send data signals to the physiological monitoring device 7.

The one or more processors 3 are used for controlling the general operations of the physiological monitoring device 7. The display/GUI 4 is for displaying various patient data and hospital or patient care information and includes a user interface implemented for allowing communication between a user and the physiological monitoring device 7. The display/GUI 4 includes, but is not limited to, a keyboard, a liquid crystal display (“LCD”), cathode ray tube (“CRT”), thin film transistor (“TFT”), light-emitting diode (“LED”), high definition (“HD”) or other similar display devices with touch screen, non-touch gesture control and/or voice control capabilities. The patient information displayed can, for example, relate to the measured physiological parameters of the patient P (e.g., blood pressure, heart-related information, pulse oximetry, respiration information, etc.) as well as information related to the transporting of the patient P (e.g., transport indicators).

The communications interface 6 allows the physiological monitoring device 7 to directly or indirectly (via, for example, the monitor mount 10) to communicate with one or more computing networks and devices. The communications interface 6 can include various network cards, interfaces, or circuitry to enable wired and wireless communications with such computing networks and devices. The communications interface 6 can also be used to implement, for example, a Bluetooth® connection, a cellular network connection, and/or a WiFi® connection. Other wireless communication connections that may be implemented using the communications interface 6 include wireless connections that operate in accordance with, but are not limited to, IEEE® 802.11 protocol, a Zigbee® protocol such as the non-limiting example of Radio Frequency for Consumer Electronics (“RF4CE”), and/or IEEE® 802.15.4 protocol.

Additionally, the communications interface 6 can enable direct device-to-device communications (e.g., messaging, signal exchange, etc.) such as from the monitor mount 10 to the physiological monitoring device 7 using, for example, a Universal Serial Bus (“USB”) connection, which may or may not be a Certified USB® connection or any of the various USB connection types trademarked by the USB Implementers Forum. The communications interface 6 can also enable direct device-to-device connection to other devices such as to a tablet, personal computer (“PC”), server, or other, similar, electronic device; or to an external storage device or memory.

The memory 8 can be a single memory or one or more memories or memory locations that include, but are not limited to, a random-access memory (“RAM”), a memory buffer, a hard drive, a solid-state drive, a database, an erasable programmable read only memory (“EPROM”), an electrically erasable programmable read only memory (“EEPROM”), a read only memory (“ROM”), a flash memory, hard disk or any other various layers of memory hierarchy. The memory 8 can be used to store any type of instructions and patient data associated with algorithms, processes, or operations for controlling the general functions and operations of the physiological monitoring device 7.

The power source 9 can include a self-contained power source such as a battery pack and/or include an interface to be powered through an electrical outlet (either directly or by way of the monitor mount 10). The power source 9 can also be a rechargeable battery that can be detached, thereby allowing for replacement. In the case of a rechargeable battery, a small built-in back-up battery (or supercapacitor) can be provided for continuous power to the physiological monitoring device 7 during battery replacement. Communication between the components of the physiological monitoring device 7 (e.g., 2, 3, 4, 6, 8, and 9) are established using an internal bus 5.

As shown in FIG. 1, the physiological monitoring device 7 is connected to the monitor mount 10 via a connection 18 that establishes a communication connection between, for example, the respective communications interfaces of the monitoring device and the monitoring mount. The connection 18 enables the monitor mount 10 to detachably secure the physiological monitoring device 7 to the monitor mount 10. In this regard, “detachably secure” means that the monitor mount 10 can secure the physiological monitoring device 7, but the physiological monitoring device 7 can be removed or undocked from the monitor mount 10 by a user when desired. The connection 18 may include, but is not limited to, a universal serial bus (“USB”) connection, parallel connection, a serial connection, coaxial connection, a High-Definition Multimedia Interface (“HDMI”)® connection, or other similar connection known in the art connecting to electronic devices. The monitor mount 10 may include one or more processors, and optionally, one or more of memory, communications interface, input/output (“I/O”) interface, and power source. In clinical settings, the patient P connected to the one or more sensors 17 that transmit physiological data to the monitoring system 1 may be under a medical procedure, for example, an IV infusion process. In one embodiment of the present disclosure, the physiological monitoring system 1 may function as an automatic pressure control device that inflates/deflates the external pressure-providing element pressurizing the IV bag, to ensure the delivery of the IV fluid in controllable manners.

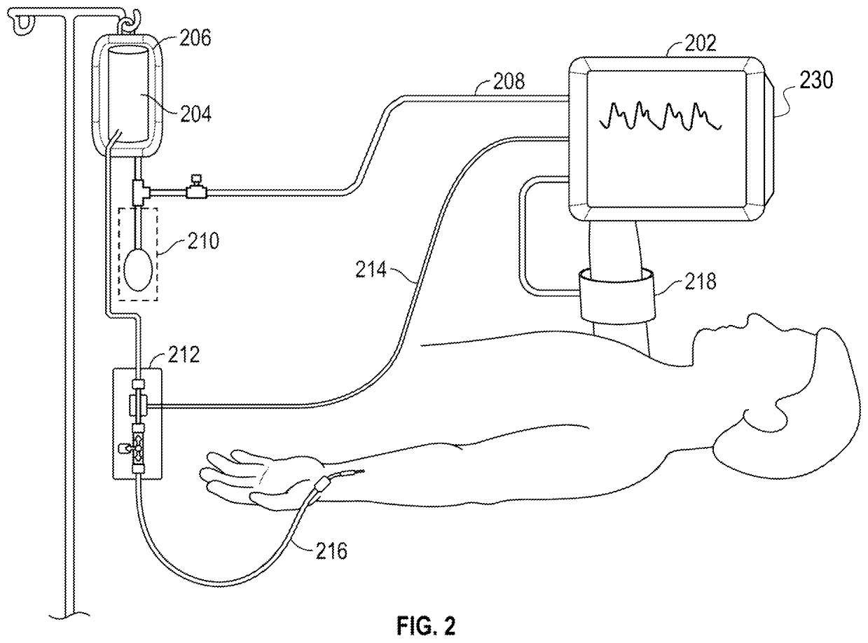

As illustrated in FIG. 2, the physiological monitoring system of FIG. 1 may include a physiological monitoring device 202 with an NIBP module 230. When an arm of the patient P is wrapped, compressed, and gradually released by an NIBP cuff 218, the blood pressure of the patient is measured, processed, and displayed by the physiological monitoring device 202. Further, the physiological monitoring device 202 may also function as the automatic pressure control device during the IV infusion process. An IV bag 204 filled with an IV fluid and pressurized by a pressure-providing element (e.g., an external pressure bag 206) is connected to the IV line 216. The IV fluid is delivered through a catheter from the IV line 216 into the vein of the patient. Optionally, a drip rate control system 212 may be connected with the physiological monitoring device 202 via an interface 214, and send data signals to the physiological monitoring device 202 during the IV infusion process.

During an NIBP measurement cycle, the NIBP module 230 of the physiological monitoring device 202 may control a pump motor and an air pump to inflate the NIBP cuff 218 to occlude the blood flow of the wrapped arm. The cuff pressure is then slowly decreased to allow arterial blood flow to return. Based on different clinical needs, the NIBP module may provide multiple working modes. For example, single measurement mode may be initiated upon the caregiver's command, where the measurement process may take less than 3 minutes and the NIBP module automatically reverts to idle thereafter. The “idle” may be referred to as the pump motor and/or the air pump is not inflating or deflating the NIBP cuff 218. During short-term or long-term interval modes, the NIBP module may automatically perform NIBP measurement cycles with pre-defined configurations (e.g., time intervals) and revert to idle mode during each time interval or when the measurement is completed. During the IV infusion process, the physiological monitoring device 202 may also function as a pressure control device that automatically monitors and controls the pressure of the external pressure bag 206. For example, when the NIBP module 230 is in the idle mode, it may be repurposed under the control of the physiological monitoring device 202. That is, the pump motor and the air pump may be connected with the external pressure bag 206 via a tubing 208 and inflate/deflate it as needed. As such, the external pressure bag 206 may apply proper pressure to the IV bag 204, thereby ensuring the fluid delivery in controllable manners. A caregiver may no longer be required to frequently monitor and/or manually adjust the pressure of the external pressure bag 206 by using the inflation bulb 210, which is burdensome and adversely affects the efficiency of the clinical workflow. The automatic pressure control device will be described in more detail as follows.

For example, during the long-term interval mode, the NIBP measurement may be configured with pre-defined intervals (e.g., from about 4 minutes to about 4 hours). During one or more intervals, the NIBP module 230 may be repurposed, under the control of the physiological monitoring device 202, to control the pressure of the external pressure bag 206. Alternatively, or additionally, during the short-term internal mode, a plurality of NIBP measurements may be automatically performed “back-to-back” within a pre-defined time period (e.g., from about 5 minutes to about 15 minutes), and the NIBP module 230 automatically reverts to idle thereafter. When the NIBP module 230 is back to idle, it may be repurposed to control the pressure of the external pressure bag 206.

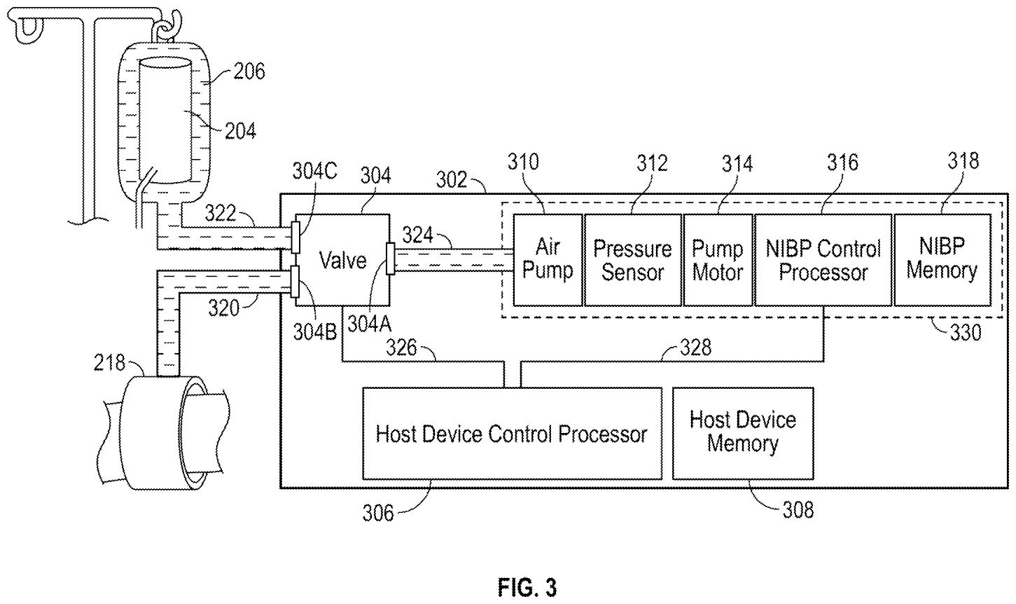

FIG. 3 is a block diagram of an example automatic pressure control system according to one embodiment of the present disclosure. The automatic pressure control device 302 may be a host device that includes, but is not limited to, the physiological monitoring devices 7, 202 as illustrated in FIGS. 1-2, a therapy device, or the like. As illustrated in FIG. 3, the automatic pressure control device 302 may include a host device control processor 306 and a host device memory 308. Further, the automatic pressure control device 302 may have an NIBP module 330 for NIBP measurements, including an air pump 310, a pressure sensor 312 and a pump motor 314. In one embodiment, the NIBP module 330 may be an original equipment manufacturer (“OEM”) module integrated into the housing of the automatic pressure control device 302. The physiological monitoring devices 7, 202 (shown in FIGS. 1-2) may similarly include the NIBP module 330. Accordingly, it may optionally include an NIBP memory 318 and an NIBP control processor 316 communicatively coupled with the host device control processor 306 via a communication interface 328.

When receiving a control signal from the host device for NIBP measurements, the NIBP control processor 316 may control the pump motor 314 and the air pump 310 to inflate/deflate the NIBP cuff 218 such that airflow between the air pump and the NIBP cuff 218 is provided at a desired air pressure value or within a desired range of air pressure values. The NIBP control processor 316 may also control the pump motor 314 and the air pump 310 to maintain the air pressure value by causing the air pump 310 to inflate/deflate the NIBP cuff 218 should a monitored value of the air pressure value be above or below a predetermined acceptable range. Should the monitored value of the air pressure value be outside the acceptable range, the air pump 310 is triggered to commence inflation or deflation depending upon whether the monitored air pressure value is higher than the maximum acceptable value or lower than the minimum acceptable value.

For example, if the air pressure value is determined to be below the minimum acceptable value, the air pump 310 may commence an inflation operation to increase the air pressure value of the NIBP cuff 218. Conversely, if the air pressure value is determined to be above the maximum acceptable value, the air pump 310 may commence a deflation operation to decrease the air pressure value of the NIBP cuff 218. As used herein, the term ‘air pressure value’ may refer to a measured and/or determined value of the air pressure within the NIBP cuff 218. Furthermore, the NIBP control processor 316 may transmit data signals generated from the pressure sensor 312 to the host device. Without limiting the scope of the present disclosure, the host device control processor 306 may directly control the air pump 310, the pressure sensor 312 and/or the pump motor 314.

The automatic pressure control device 302 may further include a controllable multi-port valve 304. A first port 304A of the valve 304 may be connected by an air hose 324 to the air pump 310 in the NIBP module 330. A second port 304B of the valve 304 may be connected by an air hose 320 to the NIBP cuff 218. and A third port 304C of the valve 304 may be connected by an air hose 322 to the external pressure bag 206.

The processor(s) may be configured to control the controllable multi-port valve 304 through an electromechanical coupling. For example, the valve 304 may be a solenoid valve with at least one input port and at least one output port, where each port may be connected with a gas/liquid hose. The valve 304 may be communicatively coupled with the host device control processor 306 via a valve control interface 326, for example, solenoid control wires. As such, the solenoid may, under the control of the host device control processor 306, energize/de-energize and change the position of a plunger in the valve. Accordingly, each of the ports may be opened up, partially restricted, or sealed off, adjusting the airflows through the air hoses 320 (to the NIBP cuff 218) and 322 (to the external pressure bag 206). The flow through the individual ports may be controlled by the host device control processor 306 to ensure a regulated airflow to the NIBP cuff 218 as well as to the external pressure bag 206.

In another embodiment, the controllable multi-port valve 304 may include more than three ports to fulfill different clinical needs. For example, the valve 304 may include one input port and three or more output ports, where the output ports are connected with one or more external pressure bags 206, one or more NIBP cuffs 218, or the like.

According to the aforementioned embodiments of the present disclosure as illustrated in FIGS. 1-3, the automatic pressure control device 302 may be a host device (e.g., the physiological monitoring device or a therapy device). Therefore, a caregiver may only need to operate via the user interface of the host device, to initiate NIBP measurements as well as the automatic pressure control of the external pressure bag 206, without plugging in additional hardware or operating on different user interfaces. For example, when the automatic pressure control device 302 is the physiological monitoring device 7 as illustrated in FIG. 1, a user may send commands and receive data for NIBP measurements via the display/GUI 4. Additionally, the user may send commands regarding the IV infusion process (e.g., configuration settings for pressure control) via the display/GUI 4.

In one embodiment of the present disclosure, a NIBP measurement cycle is initiated. The host device control processor 306 may transmit a first control signal to the controllable multi-port valve 304 via the valve control interface 326, and control the valve 304 to allow an airflow between the air hose 320 to the NIBP cuff 218 and the air pump 310, while blocking the airflow through the air hose 322 to the external pressure bag 206. The host device control processor 306 may further control the NIBP module 330 to inflate/deflate the NIBP cuff, either directly or indirectly via the NIBP control processor 316 and the communication interface 328. The pressure sensor 312 in the NIBP module 330 may include a pressure transducer and an oscillometric channel, that monitor the pressure of the cuff, convert the pressure to an analog signal, and detect small oscillometric waveforms resulting from the patient arterial pulses and manifest as a blood pressure measurement for the display/GUI 4.

While the NIBP module 330 is in the idle mode, the pump motor 314 and the air pump 310 stop inflating/deflating the NIBP cuff. When this occurs, the host device control processor 306 may transmit a second control signal to the valve 304 and allow an airflow between the air pump 310 and the air hose 322 to the external pressure bag 206, while blocking the airflow through the air hose 320 to the NIBP cuff 218. Accordingly, the pressure sensor 312 in the NIBP module 330 may automatically monitor the bag pressure, and the air pump 310 may inflate/deflate it as needed.

The present disclosure may avoid additional hardware installment (e.g., automatic or manual pump) for controlling the pressure of the external pressure bag 206. By utilizing the NIBP module 330 and switching the airflow paths by automatically controlling the valve 304, the external pressure bag 206 may be inflated/deflated as needed when the NIBP module 330 is in the idle mode. As soon as the initial inflation of the external pressure bag 206 is completed prior to or in the beginning of the IV infusion process, it may only require a short time period (e.g., from about 2 to about 15 seconds, from about 1 to about 5 seconds, from about 1 to about 15 seconds, from about 1 to 20 seconds, from about 2 to about 5 seconds) to regularly inflate/deflate the bag 206, which will not influence NIBP measurements. Additionally, the frequent monitoring and manual adjustment of the external pressure bag 206 by caregivers is avoided, which facilitates the clinical workflow.

To further ensure safety in clinical use, the automatic pressure control device 302 may further include one or more mechanisms to ensure the valve 304 adjusts the airflow under the control of the host device control processor 306. For example, when a solenoid valve is used, at least one of a mechanical contact switch, an optical sensor, or a magnetic/proximity sensor may be used to monitor the position of the solenoid. These mechanisms may ensure the solenoid is energized/deenergized properly, under the control of the host device control processor 306, to correctly switch the airflow paths through the air hoses 320 and 322 without opening up or sealing off both airflow paths simultaneously. In another embodiment of the present disclosure, the position of the solenoid plunger in the valve may be periodically monitored to ensure it is correctly positioned. The monitoring period may be, e.g., every 0.1-0.5 seconds, every 0.1-1 seconds, every 0.1-2 seconds.

In one embodiment of the present disclosure, the automatic pressure control device 302 may store algorithms for determining the priority of clinical events and automatically controlling the controllable multi-port valve 304. For example, when the valve 304 allows the airflow between the air pump 310 and the air hose 322 to the external pressure bag 206, a caregiver initiates a single NIBP measurement cycle. The automatic pressure control device 302 may determine this clinical event (e.g., the NIBP measurement) has a higher priority, which causes the host device control processor 306 to control the valve 304 to allow the airflow into the NIBP cuff 218. Accordingly, the inflation/deflation of the external pressure bag 206 is paused and the NIBP measurement is timely initiated as requested by the caregiver.

The pressure sensor 312 may continuously monitor the pressure of the external pressure bag 206 once air is allowed to flow through the air hose 322 to the bag. Alternatively, the pressure sensor 312 may monitor the pressure in pre-configured time intervals (e.g., from about 0.1 to about 1 second). In certain embodiments, the pre-configured time period ranges from about 0.05 to about 2, or from about 0.1 to about 1 or from about 0.1 to 1 seconds. During the IV infusion process, the pressure provided by the external pressure bag 206 may need to be adjusted due to the volume decrease of the IV bag 204.

When the pressure value of the external pressure bag 206 reduces below a predetermined minimum acceptable value, the automatic pressure control device 302 may control the pump motor 314 and the air pump 310 to inflate the external pressure bag via the valve 304 and the air hose 322. Likewise, when the pressure value of the external pressure bag 206 is above a predetermined maximum value, the automatic pressure control device 302 may control the pump motor 314 and the air pump 310 to deflate the bag. As such, the pressure delivered by the external pressure bag may be automatically adjusted to compensate for changes in the bag contents and maintain a regulated rate of fluid delivery from the IV bag 204. The monitored pressure value of the external pressure bag 206 may be stored in the automatic pressure control device 302 (e.g., NIBP memory 318 or host device memory 308), or transmitted to other external devices or networks.

When the pressure value of the external pressure bag 206 is outside the predetermined acceptable range, the automatic pressure control device 302 may generate at least one or more signal(s) for visual and/or audible alarms. The alarm(s) may be generated or displayed on the automatic pressure control device 302. Further, the signal for generating alarm(s) may be transmitted to external devices or networks, for example, a central station, a wearable device worn by the caregiver, or updated in the electronic medical record (“EMR”) of the patient.

The automatic pressure control device 302 may store algorithms of correlating the monitored pressure of the external pressure bag 206 with the parameters during the IV infusion process (e.g., volume of the IV bag 204, flow rate and/or data associated with the optional drip rate control system 212). For example, the automatic pressure control device 302 may store algorithms of correlating the volume of the fluid flowing out of the IV bag 204 with the volume and/or pressure of the air sent into the external pressure bag 206. Alternatively, or additionally, the automatic pressure control device 302 may store algorithms of correlating the flow rate of the fluid out of the IV bag 204 with an air flow rate from the third port 304C through the air hose 322 to the external pressure bag 206.

The initiating volume of the IV bag may be entered by the caregiver via the GUI of the automatic pressure control device 302. The flow rate may be monitored by the optional drip rate control system 212 that is communicatively coupled with the automatic pressure control device 302 (e.g., via the interface 214 as illustrated in FIG. 2). Accordingly, the automatic pressure control device 302 may monitor or estimate the consumption of the IV fluid.

When the IV bag is approaching emptiness or needs replacement, the automatic pressure control device 302 may generate signals for visual and/or audible alerts. The alerts may be displayed on the automatic pressure control device 302. Alternatively, the signals for generating alerts may be transmitted to other external devices or networks. The pressure applied to the external pressure bag 206 may be configured automatically based on physiological information of the patient and/or other parameters used in the IV infusion process. The parameters may include, but are not limited to, volume of the IV bag, drip rate, and types of nutrients/medications dissolved in the IV fluid.

Additionally, or alternatively, the pressure may be configurable by the caregiver via the user interface. Based on patient information (e.g., weight, age, gender), and/or types of NIBP cuff (e.g., adult, pediatric, neonate) worn by the patient, the automatic pressure control device 302 may automatically configure a starting pressure applied to the external pressure bag when the IV infusion process initiates. For example, an adult may have a different starting pressure compared with a non-adult (e.g., pediatric, neonate). During the IV infusion, the automatic pressure control device 302 may automatically control the pressure provided by the external pressure bag, thereby ensuring a regulated flow rate suitable for the patient.

As described above, the automatic pressure control device and the NIBP module are in the same housing as the host device. Alternatively, or additionally, a part or the whole of the automatic pressure control device and/or the NIBP module 330 may be in separate housings. For example, the automatic pressure control device may be in a separate housing from the NIBP module 330 and the host device. Alternatively, the automatic pressure control device may be in the same housing as the NIBP module, while being separated from the host device.

In one embodiment of the present disclosure, the automatic pressure control device may be in forms of a pod, a module, or the like. It may function as an external patient front-end device that is communicatively coupled with the host device via wired or wireless connections. The communications between the automatic pressure control device and its host device may follow a communication protocol. For example, lower levels of the communication may use a Universal Serial Bus (“USB”) communication protocol that enables a hub structure, detection of the attached devices, and a means of controlling power to the automatic pressure control device.

The automatic pressure control device may be able to “self-describe” its data by providing metadata. Within a standard framework for physiological parameters (e.g., blood pressure, heart rate) and/or other parameters used in the IV infusion process, the automatic pressure control device may be configured to instruct the host device how its data is formatted, how often it is updated, how it should be displayed, and how it should be labeled. These items are described by the meta-data that is transmitted by the automatic pressure control device to the host device.

The automatic pressure control device may be a “plug-and-play” device. The automatic pressure control device and the host device may be connected and communicate via a cable connector, a rack connector, or other types of connectors compatible to the external patient front-end device. Alternatively, the automatic pressure control device may be an internal patient front-end device integrated into its host device. The host device does not require restart upon being connected to the automatic pressure control device to communicatively interface with the pressure control device. Furthermore, a benefit of the external patient front-end communication protocol between the automatic pressure control device and its host device is that software in the pressure control device can be changed without changing the software in the host device.

Optionally, an external patient front-end translator may further be provided as an interface between the automatic pressure control device and another electronic device (e.g., the host device) which does not recognize the communication protocol compatible to the pressure control device. The translator may convert the data generated by the automatic pressure control device in one communication protocol into a format of another communication protocol used by the host device. Accordingly, it enables the communication between the automatic pressure control device and another device, even when they recognize different communication protocols. In one embodiment, the external patient front-end translator may include one or more processors—e.g., one or more central processing units (“CPUs”), field-programmable gate arrays (“FPGAs”), application-specific integrated circuits (“ASICs”), digital signal processors (“DSPs”), etc.

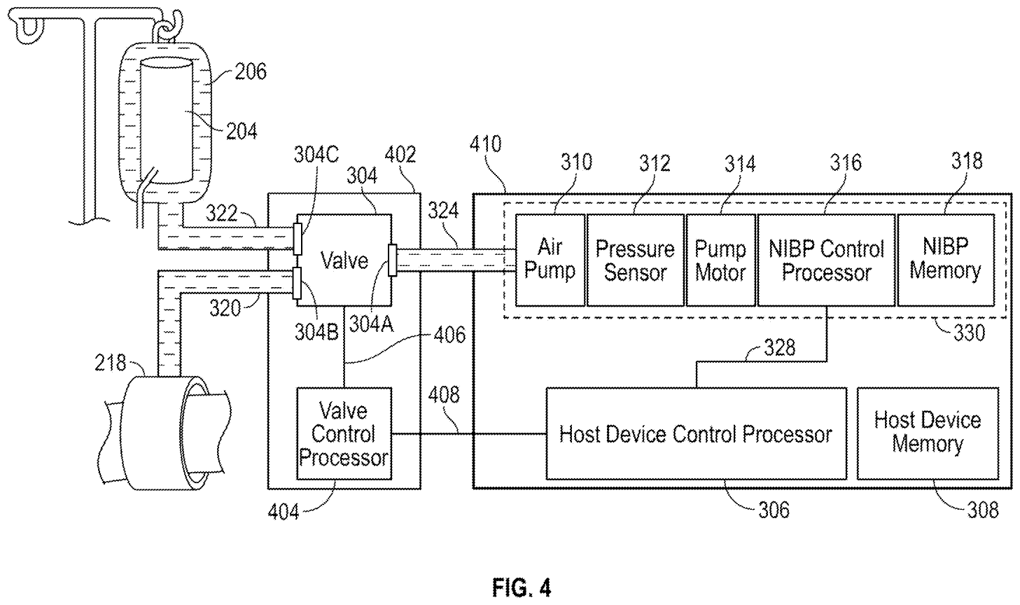

Without limiting the scope of the present disclosure, FIGS. 4-7 illustrate different embodiments of the automatic pressure control device in the forms of a pod, a module, or the like. As used herein, the term “pod” or “module” refers to hardware, firmware, and/or software that individually and/or collectively provide a specified functionality. For example, the NIBP module 330 is shown in FIG. 4 as including air pump 310, pressure sensor 312, pump motor 314, NIBP control processor 316, and NIBP memory 318, which collectively function together to effect inflation or deflation upon a determination that a monitored pressure is at a predetermined pressure value or outside of a range of acceptable pressure values. The automatic pressure control device functions as the external patient front-end device that is communicatively coupled with a host device, with more details described as follows.

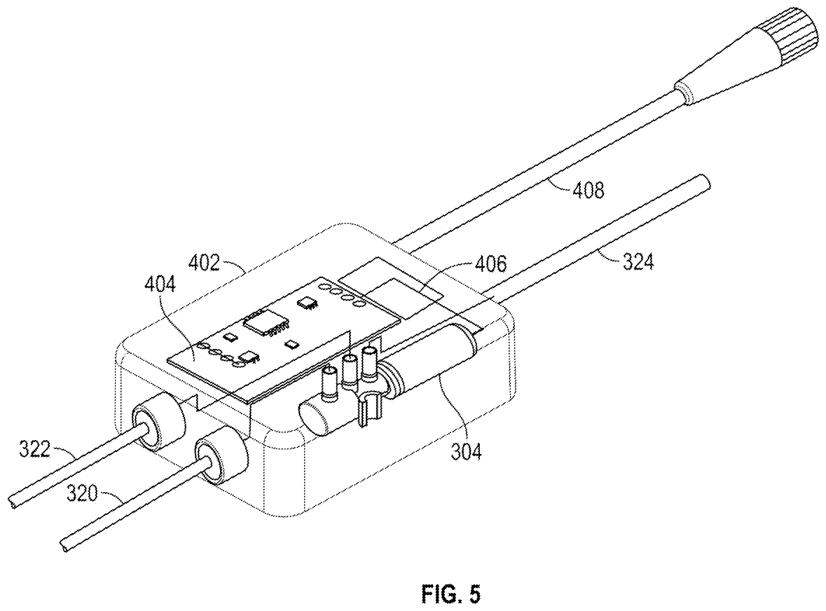

FIG. 4 is a block diagram of an example automatic pressure control device in connection with its host device, and FIG. 5 is a schematic diagram of the example automatic pressure control device in accordance with FIG. 4. As illustrated, the automatic pressure control device 402 is in a separate housing from its host device 410, while they are communicatively coupled via an external host communications interface 408. The automatic pressure control device 402 may include a controllable multi-port valve 304 and a valve control processor 404 (mounted to a printed circuit board, or “PCB”) that is connected to the valve 304 via a valve control interface 406. Optionally, the valve control processor 404 may also function as an interface processor, to communicate with the host device via the external host communications interface 408.

When an NIBP measurement cycle is initiated, the host device control processor 306 may send a first control signal to the automatic pressure control device 402 via the external host communications interface 408. Accordingly, the valve control processor 404 may control the ports 304A and 304B of the valve 304 to adjust the airflow between the air pump 310 and the air hose 320 to the NIBP cuff.

When the NIBP module 330 is in the idle mode for NIBP measurements, the host device control processor 306 may send a second control signal to the automatic pressure control device 402 via the external host communications interface 408. Accordingly, the valve control processor 404 may control the ports 304A and 304C of the valve 304 to adjust the airflow between the air pump 310 and the air hose 322 to the external pressure bag 206. Under the control of the host device control processor 306, the NIBP module 330 may be repurposed. That is, the pressure sensor 312 of the NIBP module 330 may monitor the pressure of the external pressure bag 206. The air pump 310 may inflate/deflate the external pressure bag 206 as needed, thereby regulating the flow rate of the IV fluid during the IV infusion process.

A caregiver may operate via the user interface of the host device 410 for NIBP measurements as well as the pressure control of the external pressure bag 206. For example, a caregiver may operate via the user interface of the host device 410 to configure the parameters for the IV infusion process and/or parameters for controlling the external pressure bag 206. Based on the received configuration settings and patient information from the host device 410, the automatic pressure control device 402 and/or the host device 410 may calculate an estimated time of IV fluid consumption and optimize the pressure applied to the external pressure bag 206.

Furthermore, the automatic pressure control device 402 and/or the host device 410 may generate signals for visual or audible alerts when the IV bag is approaching emptiness. The automatic pressure control device 402 and/or the host device 410 may also generate alarm signals when the monitored pressure of the external pressure bag is outside a predetermined acceptable range. When the automatic pressure control device 402 generates alert or alarm signals, they may be transmitted to the host device 410 in a communication protocol compatible with the automatic pressure control device 402.

FIG. 5 is a schematic diagram of the example automatic pressure control device. In the form of a pod, the automatic pressure control device 402 may function as the external patient-front end device that is communicatively coupled with a host device via the external host communications interface 408. In accordance with FIG. 4, the automatic pressure control device 402 may include a valve control processor 404 controlling a controllable multi-port valve 304 via a valve control interface 406. The first, second, and third ports of valve 304 may be connected by the air hose 324 to the air pump of the NIBP module, by the air hose 320 to the NIBP cuff and by the air hose 322 to the external pressure bag, respectively.

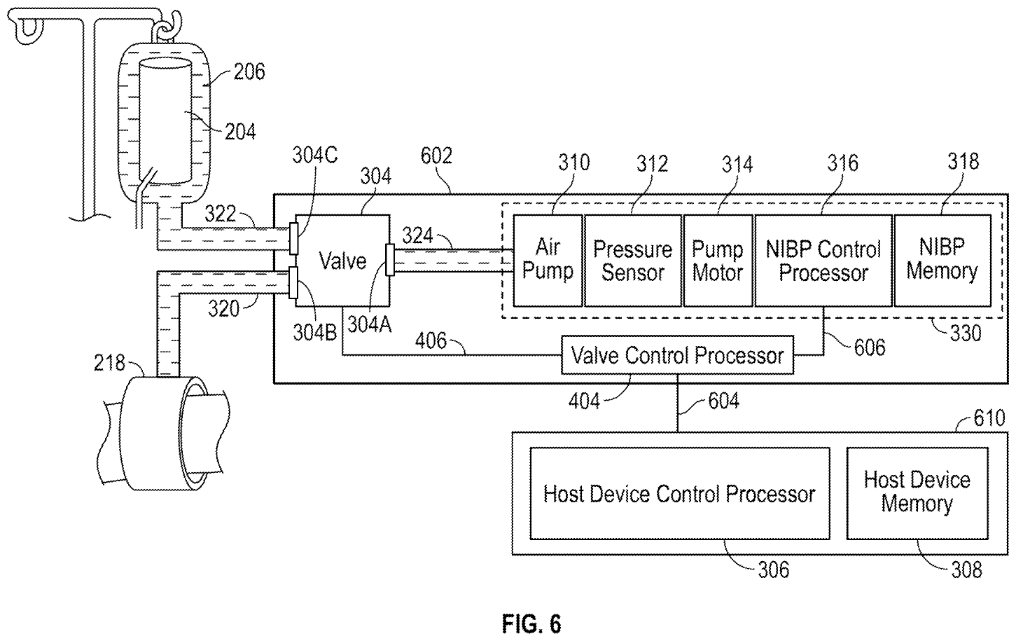

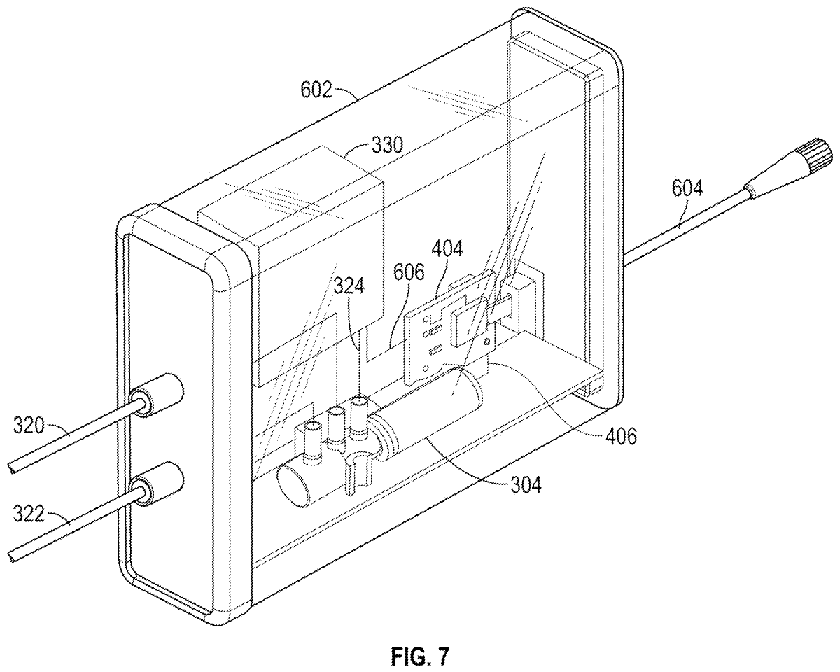

FIG. 6 is a block diagram of another example automatic pressure control device in connection with a host device according to one embodiment of the present disclosure. FIG. 7 is a schematic diagram of the example automatic pressure control device in accordance with FIG. 6. As illustrated, the automatic pressure control device 602 may be in the same housing as the NIBP module 330. The automatic pressure control device 602, with NIBP-controlling function and pressure-controlling function for IV infusion, may include a controllable multi-port valve 304, a valve control processor 404, and the NIBP module 330. The automatic pressure control device 602 may be communicatively coupled with a host device 610 via an internal host communications interface 604.

A caregiver may operate via the user interface of the host device 610 for NIBP measurements as well as the pressure control of the external pressure bag 206. When a NIBP measurement cycle is initiated, the host device control processor 306 may send a first control signal to the automatic pressure control device 402 via the internal host communications interface 604. Accordingly, the valve control processor 404 may control the first port 304A and the second port 304B of the valve 304 to adjust the airflow between the air pump 310 and the air hose 320 to the NIBP cuff 218. The valve control processor 404 may also control the NIBP module 330 to inflate/deflate the NIBP cuff 218 as needed and acquire pressure data, either directly or indirectly via the NIBP control processor 316 and the communication interface 606. The acquired data for NIBP measurements may be transmitted by the automatic pressure control device 602 in a communication protocol compatible with the device, subsequently processed and displayed by the host device 610.

When the NIBP module 330 is in the idle mode for NIBP measurements, the host device control processor 306 may send a second control signal to the automatic pressure control device 602 via the internal host communications interface 604. Accordingly, the valve control processor 404 may control the first port 304A and the third port 304C of the valve 304 to adjust the airflow between the air pump 310 and the air hose 322 to the external pressure bag 206. Under the control of the host device control processor 306 and/or the valve control processor 404, the NIBP module may be repurposed. That is, the pressure sensor 312 in the NIBP module 330 may monitor the pressure of the external pressure bag 206. The air pump 310 may inflate/deflate the pressure bag as needed, thereby regulating the flow rate of the IV fluid during the IV infusion process.

A caregiver may further operate via the user interface of the host device 610 to configure the parameters for the IV infusion process and/or parameters for pressure control of the external pressure bag 206. Based on the received configuration settings and patient information from the host device 610, the automatic pressure control device 602 and/or the host device 610 may calculate an estimated time of IV fluid consumption and optimize the pressure applied to the external pressure bag. Furthermore, the automatic pressure control device 602 and/or the host device 610 may generate signals for visual or audible alerts when the IV bag is approaching emptiness. The automatic pressure control device 602 and/or the host device 610 may also generate alarm signals when the monitored pressure of the external pressure bag is outside a predetermined acceptable range. When the automatic pressure control device 602 transmits data signals (e.g., alert or alarm signals), they may be transmitted to the host device 610 in the communication protocol compatible with the automatic pressure control device 602.

FIG. 7 illustrates a schematic diagram of the example automatic pressure control device in accordance with FIG. 6. The module structure of the automatic pressure control device 602 allows it to be detachably secured in and/or electrically connected with a module rack. For example, one or more modules may be slid in and out of the rack, while the rack being electronically connected with a host device. The automatic pressure control device 602 may function as the external patient-front end device that is communicatively coupled with a host device via the internal host communications interface 604. In accordance with FIG. 6, the automatic pressure control device 602 may include a valve control processor 404 (mounted to a printed circuit board, or “PCB”) controlling a controllable multi-port valve 304 via a valve control interface 406. The first, second, and third ports of the valve 304 may be connected with the air hose 324 to the air pump of the NIBP module, the air hose 320 to the NIBP cuff and the air hose 322 to the external pressure bag, respectively.

According to the aforementioned embodiments with reference to FIGS. 4-7, the automatic pressure control device housed in a separate housing from the host device allows simplified designs and structures. Additionally, the electromechanically controlled valve is in a separate housing from the main electronic components of the host device. The interference caused by any potential heat or electromagnetic signals generated by the controllable multi-port valve may be reduced.

When the automatic pressure control device functions as the external patient front-end device, a caregiver can simply “plug-and-play”, allowing it to be communicatively coupled with the host device without restarting the host device. Furthermore, the software in the automatic pressure control device may be updated without changing the software in the host device.

Without limiting the scope of the present disclosure, the automatic pressure control device 402 (FIGS. 4-5) and the automatic pressure control device 602 (FIGS. 6-7) may optionally include one or more of memory, communications interface, I/O interface, user interface, power source. As such, the automatic pressure control device may provide more flexibility in e.g., data storage, user operation and adaptability in hospital infrastructure.

For example, the automatic pressure control device may optionally include memory, which may be a single memory or one or more memories or memory locations. The memory can be used to store acquired physiological data, any type of instructions associated with algorithms, processes, or operations for controlling the general functions and operations of the automatic pressure control device.

The automatic pressure control device may optionally include one or more communications interfaces that allow the device to communicate with the host device, computing networks and other external devices. The communications interface may include various network cards, interfaces, or circuitry to enable wired and wireless communications with such computing networks and devices. The communications interface can also be used to implement, for example, a Bluetooth® connection, a cellular network connection, and a WiFi® connection. Other wireless communication connections implemented using the communications interface include wireless connections that operate in accordance with, but are not limited to, IEEE® 802.11 protocol, a Zigbee® protocol such as the non-limiting example of Radio Frequency For Consumer Electronics (“RF4CE”) protocol, and/or IEEE® 802.15.4 protocol. The communications interface may also enable direct device-to-device communications (e.g., signal exchange) such as between the host device and the automatic pressure control device using, for example, a USB connection, coaxial connection, or other similar electrical connection. The communications interface may enable direct device-to-device communication to other external devices. The external devices may be a tablet, PC, or similar electronic device, or alternatively an external storage device or memory.

The automatic pressure control device may optionally include an I/O interface for enabling the transfer of information between the device and other external devices. The I/O interface may include, but is not limited to, a universal serial bus (“USB”) connection, parallel connection, a serial connection, coaxial connection, or other known connection in the art connecting to external devices.

The automatic pressure control device may optionally include a user interface, including but not limited to GUI, buttons, switches, lights, or the like. The user interface on the automatic pressure control device may provide more flexibility even when the automatic pressure control device is not in proximity to the host device. A user may send commands via the user interface on the automatic pressure control device for NIBP measurements and/or pressure control of the external pressure bag for IV infusion. Furthermore, the user may receive NIBP measurement data, alert, or alarm via the user interface of the automatic pressure control device.

The automatic pressure control device may optionally include a power source, which may be a self-contained power source such as a battery pack and/or include an interface to be powered through an electrical outlet (either directly or by way of other external devices). The power source can also be a rechargeable battery that can be detached allowing for replacement.

The present disclosure further provides a computer-implemented method for pressure control by one or more processors. Instructions for performing the method may be stored on a non-transitory computer-readable medium to be executed by one or more processors controlling specialized hardware. The specialized hardware may include, without limitation, an air pump, a controllable multi-port valve, a pressure-providing element (such as an IV pressure bag) pressurizing a fluid container (such as a compressible bag of IV fluid), an NIBP cuff, and one or more sensors.

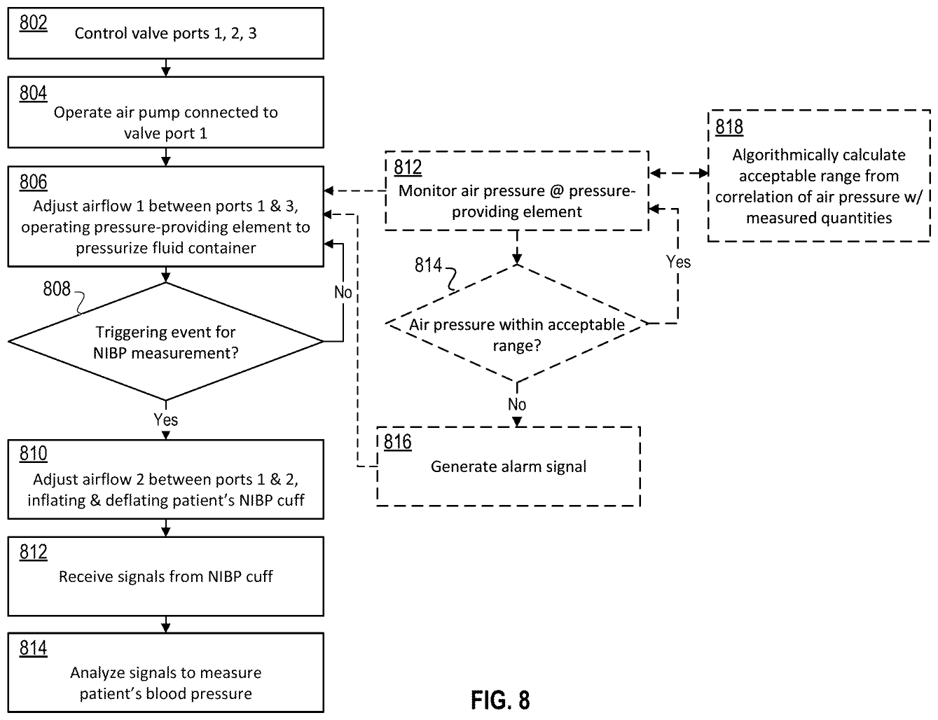

FIG. 8 is a flowchart of instructions for automatic pressure control according to one embodiment of the present disclosure. The processor(s) may cause an automatic pressure control device or system to perform step 802 (controlling a first port, a second port, and a third port of a controllable multi-port valve) and step 804 (operating an air pump connected to the first port), thereby facilitating step 806 (adjusting a first airflow between the first port and the third port to operate a pressure-providing element pressurizing a fluid container). At some point, the processor(s) may encounter decision branch 808 (a triggering event for a blood pressure measurement has or has not been recognized). As long as no such triggering event is recognized, the processor(s) continue step 806 of adjusting the first airflow. However, upon recognizing a triggering event for a blood pressure measurement, the processor(s) commence step 810 (adjusting a second airflow between the first port and the second port to inflate, then deflate, an NIBP cuff worn by a patient), step 812 (receive signals from an NIBP sensor associated with the NIBP cuff, and step 814 (analyze the signals to measure the patient's blood pressure). While NIBP measurement steps 810, 812, and 814 are performed, the device or system may interrupt IV bag pressurizing step 806, or may continue it. If IV bag pressurizing step 806 is interrupted for the NIBP measurement, it may be recommenced during or after any or all of the NIBP measurement steps 810, 812, or 814.

Blood pressure measurement may be initiated by any of a variety of triggering events: for example, a local time (“measure blood pressure at 10 PM”); a time interval since a previous event sensed by the processor (“measure blood pressure at one-hour intervals”); a volume of fluid in the fluid container (“measure blood pressure after half the IV fluid has been administered”); a signal from a sensor monitoring a physiological parameter of the patient (“measure blood pressure if the patient's heart rate or temperature change by more than a predetermined amount”); or a command entered by a caregiver. In some embodiments, the caregiver may enter the command to schedule the NIBP measurement for a later time, or may enter the command remotely, for example from a central station or a wearable device.

In some embodiments, the processor(s) may perform step 812 of monitoring an air pressure value at or near the pressure-providing element. Step 806 of controlling the valve to adjust the first airflow may be started, stopped, or modified in response to the air pressure value. For example, as the contents of the IV bag flow out into the patient's body, the air pressure at the pressure-providing element decreases if the first airflow is constant. If the processor(s) sense such a decrease during their monitoring, they may increase the first airflow in response, thus preventing an unwanted reduction in IV flow rate.

In some embodiments, while performing step 812 of monitoring the air pressure value, the processor(s) may compare the monitored values with a predetermined acceptable range of values, for example a minimum value and a maximum value. At decision branch 814, the processor(s) determine whether the air pressure value is inside or outside the predetermined acceptable range. If not, they continue monitoring (step 812) and adjusting the first airflow (step 806). However, if the air pressure is outside the predetermined acceptable range, the processor(s) may cause the device or system to perform step 816 (generating an alarm signal).

In some embodiments, the processor(s) may perform step 818 of applying an algorithm to the data acquired from air-pressure monitoring step 812. The algorithm may correlate the air pressure value to a volume of fluid in the fluid container, a flow rate of fluid from the fluid container, data from a drip rate control system, or a combination thereof. The acceptable range for decision point 814, and/or parameters of the airflow-adjusting step 806, may be dependent on the results of the algorithm-applying step 818.

A variety of method embodiments may be realized in which the processor(s) may control a controllable multi-port valve. One port of the valve may be connected with an air pump. The other two ports of the valve may be connected with an air hose to the NIBP cuff and an air hose to the external pressure bag that pressurizes the IV bag for IV infusion, respectively. When a NIBP measurement cycle is initiated, the one or more processors may control the valve to allow an airflow between the air pump and the NIBP cuff, while blocking an airflow to, through, or from the external pressure bag. The processors may further control the air pump to inflate/deflate the NIBP cuff, thereby measuring the blood pressure of the patient wearing the NIBP cuff. When the NIBP measurement cycle is not taking place, the one or more processors may control the valve to allow an airflow between the air pump and the external pressure bag, while blocking the air to, through, or from the NIBP cuff. Under the control of the processors, the external pressure bag may be automatically inflated/deflated as needed, thereby providing pressure to the IV bag and ensuring a regulated IV fluid delivery to the patient.

Each one of the one or more processors described in the present disclosure, for example, processor(s) 3 illustrated in FIG. 1 and host device control process processor 306 and the NIBP control processor 316 that are illustrated in FIG. 3 and the valve control processor 404 illustrated in FIGS. 4-7 can be, but are not limited to, a central processing unit (“CPU”), a hardware microprocessor, a multi-core processor, a single core processor, a field programmable gate array (“FPGA”), a microcontroller, an application specific integrated circuit (“ASIC”), a digital signal processor (“DSP”), or other similar processing devices capable of executing any type of instructions, algorithms, or software for controlling the operation and performing the functions of the corresponding devices.

Furthermore, the one or more processors described in the present disclosure may be implemented on the same circuit board or independent circuit boards as one or more integrated circuit (“IC”), an application specific integrated circuit (“ASIC”), or large-scale integrated circuit (“LSI”), system LSI, super LSI, or ultra-LSI components which perform part or all of the functions described in the present disclosure.

The present disclosure may be implemented as a non-transitory computer-readable recording medium having recorded thereon a program embodying methods/algorithms for instructing the processor to perform the methods/algorithms. The non-transitory computer-readable recording medium can be, for example, a read-only memory in compact disc form (CD-ROM), digital video disc or digital versatile disc (DVD), Blu-ray Disc®, or an electronic memory device. For example, referring to FIG. 1, the programs or algorithms can be stored on a non-transitory computer-readable medium for causing a computer, such as the processor(s) 3 to perform the steps or method described herein.

The computer programs, which can also be referred to as programs, software, software applications, applications, components, or code, include machine instructions for a programmable processor, and can be implemented in a high-level procedural language, an object-oriented programming language, a functional programming language, a logical programming language, or an assembly language or machine language. The term computer-readable recording medium refers to any computer program product, apparatus, or device, such as a magnetic disk, optical disk, solid-state storage device, memory, and programmable logic devices (“PLDs”), used to provide machine instructions or data to a programmable data processor, including a computer-readable recording medium that receives machine instructions as a computer-readable signal.

Each of the elements of the present disclosure may be configured by implementing dedicated hardware or a software program on a memory controlling a processor to perform the functions of any of the components or combinations thereof. Any of the components may be implemented as a CPU or other processor reading and executing a software program from a recording medium such as a hard disk or a semiconductor memory or network or cloud-based programs.

It is also contemplated that the implementation of the components of the present disclosure can be done with any newly arising technology that may replace any of the above implementation technologies.

By way of non-limiting example, a computer-readable medium can comprise memory circuits, chips, or modules such as dynamic random-access memory (“DRAM”), other types of random-access memory (“RAM”), read-only memory (“ROM”), electrically-erasable programmable read-only memory (“EEPROM”), read-only memory on a compact disc (“CD-ROM”); other optical or magnetic storage devices, or any other medium that can be used to carry or store desired computer-readable program code in the form of instructions or data structures and that can be accessed by a general-purpose or special-purpose computer, or a general-purpose or special-purpose processor. “Disk” or “disc,” as used herein, include compact disc (“CD”), laser disc, optical disc, digital versatile disc (“DVD”), floppy disk, Blu-ray Disc®, and similar storage schema where data may be reproduced magnetically or optically. Combinations of the above are also included within the scope of computer-readable media.

Use of the phrases “capable of,” “capable to,” “operable to,” or “configured to” in one or more embodiments, refers to some apparatus, logic, hardware, and/or element designed in such a way to enable use of the apparatus, logic, hardware, and/or element in a specified manner. The subject matter of the present disclosure is provided as examples of apparatus, systems, methods, circuit, and programs for performing the features described in the present disclosure. However, further features or variations are contemplated in addition to the features described above. It is contemplated that the implementation of the components and functions of the present disclosure can be done with any newly arising technology that may replace any of the above implemented technologies.

Additionally, the above description provides examples, and is not limiting of the scope, applicability, or configuration set forth in the claims. Changes may be made in the function and arrangement of elements discussed without departing from the spirit and scope of the disclosure. Various embodiments may omit, substitute, or add various procedures or components as appropriate. For instance, features described with respect to certain embodiments may be combined in other embodiments.

Various modifications to the disclosure will be readily apparent to those skilled in the art, and the generic principles defined herein may be applied to other variations without departing from the spirit or scope of the present disclosure. Throughout the present disclosure the terms “example,” “examples,” or “example” indicate examples or instances and do not imply or require any preference for the noted examples. Thus, the present disclosure is not to be limited to the examples and designs described herein but is to be accorded the widest scope consistent with the principles and novel features disclosed.

What is claimed is:

1. A pressure control device, comprising:
a controllable multi-port valve with a first port, a second port, and a third port, wherein:
the first port is connected with an air pump;
the second port is connected with a non-invasive blood pressure (“NIBP”) cuff; and
the third port is connected with a pressure-providing element pressurizing a fluid container; and
one or more processors configured to control the controllable multi-port valve to adjust airflows through the first port, the second port, and the third port, thereby regulating a first air pressure in the NIBP cuff and a second air pressure in the pressure-providing element.

2. The pressure control device according to claim 1, wherein:
the one or more processors comprise at least one of a host device processor or a valve control processor.

3. The pressure control device according to claim 1, wherein:
the one or more processors monitor an air pressure value of the pressure-providing element while controlling the valve.

4. The pressure control device according to claim 3, wherein the one or more processors are further programmed to:
determine whether the air pressure value of the pressure-providing element is outside a predetermined acceptable range, and
generate an alarm signal when the air pressure value of the pressure-providing element is determined to be outside the predetermined acceptable range.

5. The pressure control device according to claim 1, further comprising:
a communications interface, wherein the pressure control device is communicatively coupled to at least one of a host device or a network via the communications interface.

6. The pressure control device according to claim 5, wherein:
the one or more processors are configured to transmit data, via the communications interface, to the at least one of the host device or the network.

7. The pressure control device according to claim 1, wherein:
when the valve is controlled to adjust the airflow between the air pump and the pressure-providing element pressurizing the fluid container, the pressure-providing element applies pressure to the fluid container.

8. The pressure control device according to claim 7, wherein:
the fluid container includes liquid used in an intravenous infusion process, and
the pressure-providing element is configured to control the liquid to flow out of the fluid container by applying the pressure to the fluid container.

9. A pressure control system, comprising:
a controllable multi-port valve with a first port, a second port, and a third port, wherein:
the first port is connected to an air pump,
the second port is connected to a non-invasive blood pressure (“NIBP”) cuff worn by a patient, and
the third port is connected to a pressure-providing element pressurizing a fluid container; and
one or more processors configured to:
non-invasively measure a blood pressure of the patient while controlling the valve to adjust an airflow between the air pump and the NIBP cuff, and
control pressure provided to the fluid container by controlling the valve to adjust an airflow between the air pump and the pressure-providing element.

10. The pressure control system according to claim 9, wherein:
the one or more processors are configured to monitor an air pressure value of the pressure-providing element while controlling the valve to allow the airflow between the air pump and the pressure-providing element.

11. The pressure control system according to claim 10, further comprising:
the one or more processors determine whether the air pressure value of the pressure-providing element is outside a predetermined acceptable range, and
the one or more processors generate an alarm signal when the air pressure value of the pressure-providing element is determined to be outside the predetermined acceptable range.

12. The pressure control system according to claim 9, further comprising:
a communications interface, wherein the pressure control device is communicatively coupled with at least one of a host device or a network.

13. The pressure control system according to claim 12, wherein:
the one or more processors are communicatively coupled to the communications interface to transmit data to the at least one of the host device or the network.

14. The pressure control system according to claim 9, wherein:
when the valve is controlled to allow the airflow between the air pump and the pressure-providing element pressurizing the fluid container, the pressure-providing element applies pressure to the fluid container.

15. The pressure control system according to claim 14, wherein:
the fluid container includes liquid used in an intravenous infusion process; and
the pressure-providing element is configured to control a flow rate of the liquid out of the fluid container by applying the pressure to the fluid container.

16. A computer-implemented method for pressure control, comprising:
controlling a first port, a second port, and a third port of a controllable multi-port valve;
operating an air pump connected to the first port;
adjusting a first airflow between the first port and the third port to operate a pressure-providing element pressurizing a fluid container;
recognizing a triggering event for a blood pressure measurement;
adjusting a second airflow between the first port and the second port to inflate, then deflate, an NIBP cuff worn by a patient;
receiving signals from an NIBP sensor associated with the NIBP cuff; and
calculating a blood pressure of the patient by analyzing the signals;
wherein the controlling of the ports, the operating of the air pump, the adjusting of the first airflow, the recognizing of the triggering event, and the adjusting of the second airflow are implemented by one or more processors.

17. The computer-implemented method for pressure control according to claim 16, further comprising:
monitoring, by the one or more processors, an air pressure value of the pressure-providing element; and
controlling the valve to adjust the first airflow in response to the air pressure value.

18. The computer-implemented method for pressure control according to claim 17, further comprising:
determining, by the one or more processors, whether the air pressure value of the pressure-providing element is inside or outside a predetermined acceptable range, and
generating, by the one or more processors, an alarm signal when the air pressure value of the pressure-providing element is determined to be outside the predetermined acceptable range.

19. The computer-implemented method for pressure control according to claim 17, further comprising:
applying an algorithm for correlating the air pressure value to at least one of:
a volume of fluid in the fluid container,
a flow rate of fluid from the fluid container, and
data from a drip rate control system;
wherein the instructions to adjust the first airflow depend partly on results of the algorithm.

20. The computer-implemented method for pressure control according to claim 16, wherein the triggering event for a blood pressure measurement comprises at least one of:
a local time;
a time interval since a previous event sensed by the processor;
a volume of fluid in the fluid container;
a signal from a sensor monitoring a physiological parameter of the patient; or
a command entered by a caregiver.

21. A non-transitory computer-readable medium encoded with instructions that, when executed, cause one or more processors to:
control a first port, a second port, and a third port of a controllable multi-port valve;
operate an air pump connected to the first port;
adjust a first airflow between the first port and the third port to operate a pressure-providing element pressurizing a fluid container;
recognize a triggering event for a blood pressure measurement;
adjust a second airflow between the first port and the second port to inflate, then deflate, an NIBP cuff worn by a patient;
receive signals from an NIBP sensor associated with the NIBP cuff; and
calculate a blood pressure of the patient by analyzing the signals.

22. The non-transitory computer-readable medium according to claim 16, further comprising instructions to:
monitor an air pressure value of the pressure-providing element; and
control the valve to adjust the first airflow in response to the air pressure value.

23. The non-transitory computer-readable medium according to claim 17, further comprising instructions to:
determine whether the air pressure value is inside or outside a predetermined acceptable range, and
generate an alarm signal when the air pressure value is determined to be outside the predetermined acceptable range.

24. The non-transitory computer-readable medium according to claim 17, further comprising an algorithm for correlating the air pressure value to at least one of:
a volume of fluid in the fluid container,
a flow rate of fluid from the fluid container, and
data from a drip rate control system;
wherein the instructions to adjust the first airflow depend partly on results of the algorithm.

25. The non-transitory computer-readable medium according to claim 16, wherein the triggering event for a blood pressure measurement comprises at least one of:
a local time;
a time interval since a previous event sensed by the processor;
a volume of fluid in the fluid container;
a signal from a sensor monitoring a physiological parameter of the patient; or
a command entered by a caregiver.
